(19) United States
(12) Patent Application Publication
(10) Pub. No.: US 2026/0284048 A1
(43) Pub. Date: Sep. 24, 2026

(54) INHIBITOR OF MEGAKARYOCYTE DIFFERENTIATION AND MATURATION

(71) Applicant: OTSUKA PHARMACEUTICAL CO., LTD., Tokyo (JP)

(72) Inventors: Emiri TAKAKI, Osaka-Shi, Osaka (JP); Kazuhide NAKAMURA, Osaka-Shi, Osaka (JP)

(73) Assignee: OTSUKA PHARMACEUTICAL CO., LTD., Tokyo (JP)

(21) Appl. No.: 19/475,211

(22) PCT Filed: May 7, 2024
(86) PCT No.: PCT/JP2024/016964

§ 371 Date: Oct. 14, 2025

(30) Foreign Application Priority Data
May 8, 2023 (JP) 2023-076589

(51) Int. Cl. A61K 31/513; A61P 37/02

(52) U.S. Cl. CPC A61K 31/513; A61P 37/02

(57) ABSTRACT

The present invention relates to a compound of formula (I) which can selectively inhibit the differentiation from hematopoietic stem cell toward megakaryocytes.

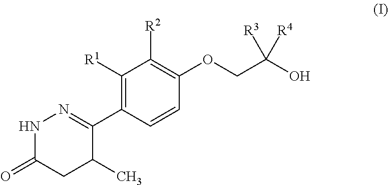

### TECHNICAL FIELD

The present invention relates to an inhibitor of megakaryocyte differentiation and maturation, comprising a 5-methyl-6-phenyl-4,5-dihydro-2H-pyridazin-3-one derivative. In more detail, it relates to an inhibitor of platelet production (for example, a medicament for treating essential thrombocythemia).

### BACKGROUND ART

Essential thrombocythemia (also referred to as primary thrombocythemia) is a disease characterized by increased platelet counts, megakaryocytic hyperplasia, and hemorrhagic or thrombotic tendencies. The symptoms and signs include muscle weakness, headache, paresthesia, hemorrhage, and erythromelalgia with digital ischemia. The prevalence rate is approximately 0.48 per 100,000 people (Non-Patent Literature 1).

Non-Patent Literature 2 classifies anagrelide which is a medicament for treating essential thrombocythemia as a phosphodiesterase 3 (PDE3) inhibitor. The literature suggests that the mechanism of anagrelide may be based on the interaction between PDE3A and Schlafen 12 (SLFN12) since the action of anagrelide inhibits megakaryocyte differentiation, which is different from other PDE3 inhibitors. However, the literature does not disclose any results of demonstrating the effect on megakaryocytes.

Patent Literature 1 discloses 5-methyl-6-phenyl-4,5-dihydro-2H-pyridazin-3-one derivatives as a medicament for treating malignant tumor.

### CITATION LIST

### Patent Literature

[PL 1] WO2017/150654

### Non-Patent Literature

[NPL 1] Orphanet Report Series; Prevalence and incidence of rare diseases: Bibliographic data, January 2022 (www.orhpa.net)
[NPL 2] Waal, L et al., Nat. Chem. Biol. 2016; 12: 102-8

### SUMMARY OF INVENTION

### Technical Problem

The purpose of the present invention may be to provide a compound which can selectively inhibit the differentiation from hematopoietic stem cell toward megakaryocytes.

### Solution to Problem

The present inventors have extensively studied to reach the above purpose, and then have found that a compound of formula (I) below has an action of promoting complex formation of PDE3A and SLFN12. In addition, they have also found that the compound inhibits megakaryocyte differentiation without affecting erythroblasts and myeloid cell differentiation from hematopoietic stem cells. Based upon the new findings, the present invention has been completed.

The present invention includes the following embodiments.

(1-1)

A pharmaceutical composition comprising a compound of formula (I):

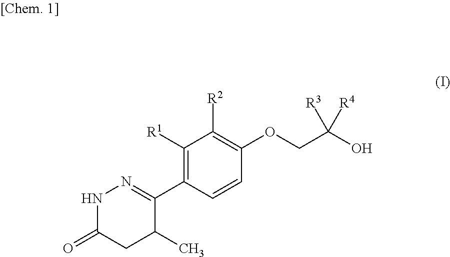

wherein R¹, R², R³, and R⁴ are independently halogen or C₁₋₃ alkyl, or a pharmaceutically acceptable salt thereof, for preventing and/or treating a symptom and/or a disease related to enhanced induction of megakaryocyte differentiation.
   (1-2)

The pharmaceutical composition of (1-1), wherein the compound of formula (I) is a compound of formula (Ia)

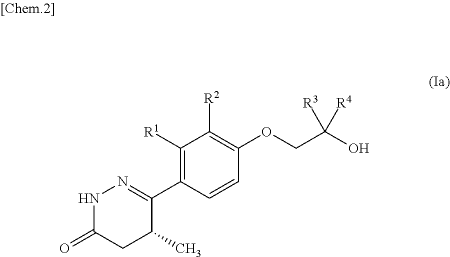

(1-3)

The pharmaceutical composition of (1-1) or (1-2), wherein R¹ and R² are fluorine, and R³ and R⁴ are methyl.

(1-4)

The pharmaceutical composition of any one of (1-1)-(1-3), wherein the compound of formula (I) is
6-(2,3-difluoro-4-(2-hydroxy-2-methylpropoxy)phenyl)-5-methyl-4,5-dihydropyridazin-3(2H)-one or
(R)-6-(2,3-difluoro-4-(2-hydroxy-2-methylpropoxy)phenyl)-5-methyl-4,5-dihydropyridazin-3(2H)-one.
   (1-5)

The pharmaceutical composition of any one of (1-1)-(1-4), wherein the symptom related to enhanced induction of megakaryocyte differentiation is increased platelet production.

(1-6)

The pharmaceutical composition of any one of (1-1)-(1-4), wherein the disease related to enhanced induction of megakaryocyte differentiation is essential thrombocythemia.

(2-1)

Use of a compound of formula (I):

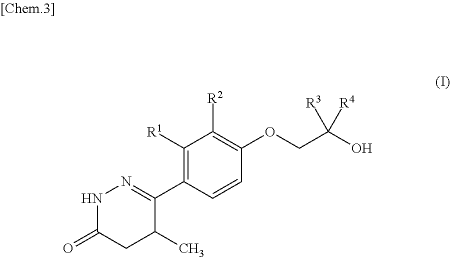

wherein R¹, R², R³, and R⁴ are independently halogen or C₁₋₃ alkyl, or a pharmaceutically acceptable salt thereof, for preventing and/or treating a symptom and/or a disease related to enhanced induction of megakaryocyte differentiation.
   (2-2)

The use of (2-1), wherein the compound of formula (I) is a compound of formula (Ia):

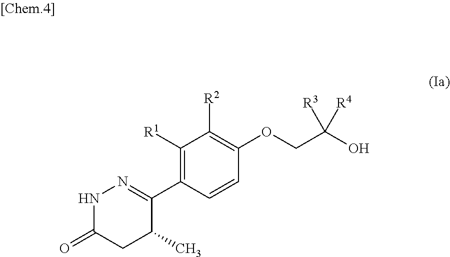

(2-3)

The use of (2-1) or (2-2), wherein the compound of formula (I) is
6-(2,3-difluoro-4-(2-hydroxy-2-methylpropoxy)phenyl)-5-methyl-4,5-dihydropyridazin-3(2H)-one or
(R)-6-(2,3-difluoro-4-(2-hydroxy-2-methylpropoxy)phenyl)-5-methyl-4,5-dihydropyridazin-3(2H)-one.
   (2-4)

The use of any one of (2-1)-(2-3), wherein the symptom related to enhanced induction of megakaryocyte differentiation is increased platelet production.

(2-5)

The use of any one of (2-1)-(2-3), wherein the disease related to enhanced induction of megakaryocyte differentiation is essential thrombocythemia.

(3-1)

A method for preventing and/or treating a symptom and/or a disease related to enhanced induction of megakaryocyte differentiation, comprising administering an effective amount of a compound of formula (T):

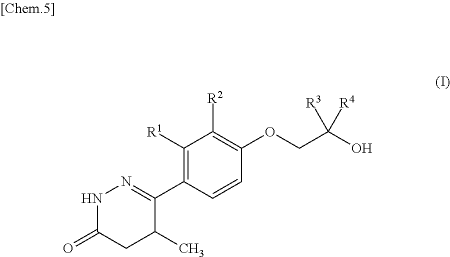

wherein R¹, R², R³, and R⁴ are independently halogen or C₁₋₃ alkyl, or a pharmaceutically acceptable salt thereof to a subject in need thereof.
   (3-2)

The method of (3-1), wherein the compound of formula (I) is a compound of formula (Ia):

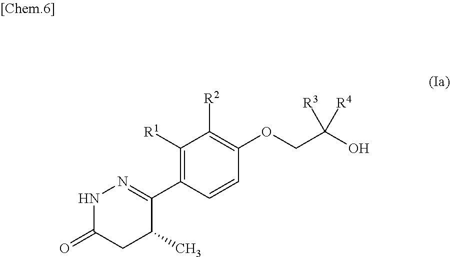

(3-3)

The method of (3-1) or (3-2), wherein the compound of formula (I) is
6-(2,3-difluoro-4-(2-hydroxy-2-methylpropoxy)phenyl)-5-methyl-4,5-dihydropyridazin-3(2H)-one or
(R)-6-(2,3-difluoro-4-(2-hydroxy-2-methylpropoxy)phenyl)-5-methyl-4,5-dihydropyridazin-3(2H)-one.
   (3-4)

The method of any one of (3-1)-(3-3), wherein the symptom related to enhanced induction of megakaryocyte differentiation is increased platelet production.

(3-5)

The method of any one of (3-1)-(3-3), wherein the disease related to enhanced induction of megakaryocyte differentiation is essential thrombocythemia.

(4-1)

A compound of formula (I):

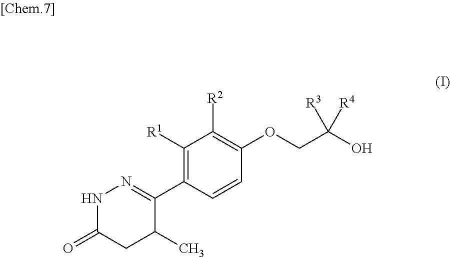

wherein R¹, R², R³, and R⁴ are independently halogen or C₁₋₃ alkyl, or a pharmaceutically acceptable salt thereof for use in preventing and/or treating a symptom and/or a disease related to enhanced induction of megakaryocyte differentiation.
   (4-2)

The compound or a pharmaceutically acceptable salt thereof of (4-1), wherein the compound of formula (I) is a compound of formula (Ta):

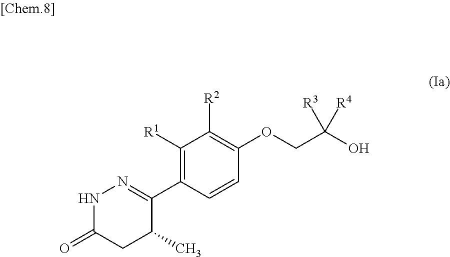

(4-3)

The compound or a pharmaceutically acceptable salt thereof of (4-1) or (4-2), wherein the compound of formula (I) is
6-(2,3-difluoro-4-(2-hydroxy-2-methylpropoxy)phenyl)-5-methyl-4,5-dihydropyridazin-3(2H)-one or
(R)-6-(2,3-difluoro-4-(2-hydroxy-2-methylpropoxy)phenyl)-5-methyl-4,5-dihydropyridazin-3(2H)-one.
   (4-4)

The compound or a pharmaceutically acceptable salt thereof of any one of (4-1)-(4-3), wherein the symptom related to enhanced induction of megakaryocyte differentiation is increased platelet production.

(4-5)

The compound or a pharmaceutically acceptable salt thereof of any one of (4-1)-(4-3), wherein the disease related to enhanced induction of megakaryocyte differentiation is essential thrombocythemia.

(5-1)

Use of a compound of formula (I):

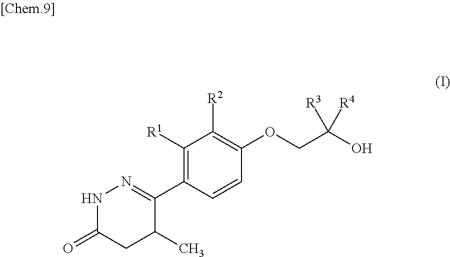

wherein R¹, R², R³, and R⁴ are independently halogen or C₁₋₃ alkyl,
or a pharmaceutically acceptable salt thereof, in the manufacture of a medicament for preventing and/or treating a symptom and/or a disease related to enhanced induction of megakaryocyte differentiation.
   (5-2)

The use of (5-1), wherein the compound of formula (I) is a compound of formula (Ia):

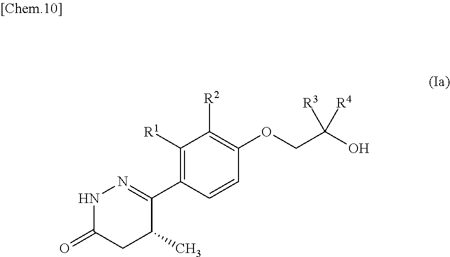

(5-3)

The use of (5-1) or (5-2), wherein the compound of formula (I) is
6-(2,3-difluoro-4-(2-hydroxy-2-methylpropoxy)phenyl)-5-methyl-4,5-dihydropyridazin-3(2H)-one or
(R)-6-(2,3-difluoro-4-(2-hydroxy-2-methylpropoxy)phenyl)-5-methyl-4,5-dihydropyridazin-3(2H)-one.
   (5-4)

The use of any one of (5-1)-(5-3), wherein the symptom related to enhanced induction of megakaryocyte differentiation is increased platelet production.

(5-5)

The use of any one of (5-1)-(5-3), wherein the disease related to enhanced induction of megakaryocyte differentiation is essential thrombocythemia.

### Effect of Invention

The present compound or a pharmaceutically acceptable salt thereof can selectively inhibit the differentiation from hematopoietic stem cell toward megakaryocytes, thus it is expected to have an effect for treating and/or preventing thrombocytosis such as essential thrombocythemia.

### BRIEF DESCRIPTION OF DRAWINGS

FIG. 1 shows the result of electrophoresis showing the formation of a complex between PDE3A and SLFN12 in Example 1.

FIG. 2 shows the result of electrophoresis showing the increase of SLFN12 protein in Example 2.

FIG. 3 is a graph which shows the effect on differentiation toward megakaryocytes in Example 3.

FIG. 4 is a graph which shows the effect on differentiation toward erythroid cells in Example 4.

FIG. 5 is a graph which shows the effect on differentiation toward myeloid cells in Example 5.

### DESCRIPTION OF EMBODIMENTS

The phrases and terms used herein are explained in detail below.

In the present specification, the “halogen” is fluorine, chlorine, bromine, or iodine. It is preferably fluorine, chlorine, or bromine, more preferably fluorine or chlorine, and even more preferably fluorine.

In the present specification, the “C₁₋₃ alkyl” is a linear or branched alkyl having 1 to 3 carbon atoms (C₁₋₃), and specific examples include methyl, ethyl, n-propyl, and isopropyl, preferably methyl.

The “subject” used herein is a vertebrate, and specific examples thereof include mammals, reptiles, and birds. Examples of mammals include humans, companion animals (for example, dogs and cats), and livestock animals (for example, cows, horses, pigs, and sheep), and preferably humans.

Each substituent in the compound of formula (I) herein (hereinafter referred to as “Compound (I)”) is explained below.

R¹, R², R³, and R⁴ in Compound (I) are independently halogen or C₁₋₃ alkyl, preferably fluorine or methyl. Or, R¹ and R² are halogen, preferably fluorine, and R³ and R⁴ are C₁₋₃ alkyl, preferably methyl.

A specific embodiment of Compound (I) is
6-(2,3-difluoro-4-(2-hydroxy-2-methylpropoxy)phenyl)-5-methyl-4,5-dihydropyridazin-3(2H)-one, or
(R)-6-(2,3-difluoro-4-(2-hydroxy-2-methylpropoxy)phenyl)-5-methyl-4,5-dihydropyridazin-3(2H)-one.

A specific embodiment of Compound (I) is
6-(2,3-difluoro-4-(2-hydroxy-2-methylpropoxy)phenyl)-5-methyl-4,5-dihydropyridazin-3(2H)-one.

A specific embodiment of Compound (I) is
(R)-6-(2,3-difluoro-4-(2-hydroxy-2-methylpropoxy)phenyl)-5-methyl-4,5-dihydropyridazin-3(2H)-one.

Compound (I) in the present invention includes a pharmaceutically acceptable salt thereof. The pharmaceutically acceptable salt thereof may be an acid addition salt, or possibly a salt with a base depending on the kinds of substituents. Examples of the “acid” here include inorganic acids such as hydrochloric acid, hydrobromic acid, nitric acid, sulfuric acid, and phosphoric acid; and organic acids such as methanesulfonic acid, p-toluenesulfonic acid, acetic acid, citric acid, tartaric acid, maleic acid, fumaric acid, malic acid, and lactic acid. Examples of the “base” include inorganic bases such as sodium hydroxide, potassium hydroxide, calcium hydroxide, sodium carbonate, potassium carbonate, sodium bicarbonate, and potassium bicarbonate; organic bases such as methylamine, diethylamine, trimethylamine, triethylamine, ethanolamine, di-ethanolamine, triethanolamine, ethylenediamine, tris(hydroxymethyl)methylamine, di-cyclohexylamine, N,N′-dibenzylethylenediamine, guanidine, pyridine, picoline, and choline; and ammonium salts and the like. The present compound may also form a salt with an amino acid such as lysine, arginine, aspartic acid, glutamic acid, and the like.

Compound (I) of the present invention also encompasses various hydrates, solvates and crystal polymorphisms of Compound (I) and salts thereof.

Compound (I) of the present invention may be in a form of co-crystal or co-crystal salt. The co-crystal or co-crystal salt means a crystalline substance composed of two or more individual solids at room temperature, said individual solids have different physical properties (e.g. structure, melting point, heat of fusion, etc.). Such co-crystals and co-crystal salts can be prepared by using known co-crystallization methods.

Compound (1) of the present invention is a known compound, which can be prepared, for example, in manner of a method disclosed in WO2017/150654.

The pharmaceutical composition comprising Compound (I) of the present invention as an active ingredient is described below.

The above pharmaceutical composition is a formulation of the present Compound (I) in the form of a general pharmaceutical composition, which is prepared with generally-used carriers, diluents and/or excipients such as fillers, bulking agents, binders, humectants, disintegrants, surfactants, and lubricants (also collectively referred to as “pharmaceutically acceptable carrier” herein).

Such pharmaceutical composition can be selected from various forms depending on the therapeutic purpose, and the typical examples thereof include tablets, pills, powders, liquids, suspensions, emulsions, granules, capsules, suppositories, and injections (liquids, suspensions, etc.).

In forming oral preparations such as tablets, a wide range of known carriers can be used. Examples of the carrier include excipients such as lactose, white sugar, sodium chloride, glucose, urea, starch, calcium carbonate, kaolin, and crystalline cellulose; binders such as water, ethanol, propanol, simple syrup, glucose solution, starch solution, gelatin solution, carboxymethylcellulose, shellac, methylcellulose, potassium phosphate, and polyvinylpyrrolidone; disintegrants such as dry starch, sodium alginate, agar powder, laminaran powder, sodium bicarbonate, calcium carbonate, polyoxyethylene sorbitan fatty acid esters, sodium lauryl sulfate, stearic acid mono-glyceride, starch, and lactose; disintegration inhibitors such as white sugar, stearin, cocoa butter, and hydrogenated oil; absorption enhancers such as quaternary ammonium base and sodium lauryl sulfate; moisturizers such as glycerin and starch; adsorbents such as starch, lactose, kaolin, bentonite, and colloidal silicate; lubricants such as purified talc, stearate, boric acid powder, and polyethylene glycol; and the like. Furthermore, flavorings, flavoring agents, sweetening agents, etc. may be added as necessary.

Furthermore, the tablets can be coated with a general coating material if necessary to prepare, for example, sugar-coated tablets, gelatin-coated tablets, enteric-coated tablets, film-coated tablets, or double-layered tablets or multilayered tablets.

In forming pills, a wide range of known carriers can be used. Examples of the carrier include excipients such as glucose, lactose, starch, cocoa butter, hydrogenated vegetable oil, kaolin, and talc: binders such as gum arabic powder, tragacanth powder, gelatin, and ethanol; disintegrants such as laminaran and agar; and the like.

In forming suppositories, a wide range of known carriers can be used. Examples of the carrier include polyethylene glycol, cocoa butter, higher alcohols, esters of higher alcohols, gelatin, and semi-synthetic glycerides.

Liquids, emulsions, and suspensions prepared as injections are preferably sterilized and made isotonic to blood. Diluents used in the liquids, emulsions, and suspensions can be any known and widely-used diluents such as water, ethanol, propylene glycol, ethoxylated isostearyl alcohol, polyoxylated isostearyl alcohol, and polyoxyethylene sorbitan fatty acid esters. In this case, these preparations may comprise sodium chloride, glucose, or glycerin in amounts sufficient to prepare an isotonic solution, and may also comprise general solubilizing agents, buffers, soothing agents, etc., and further, if necessary, coloring agents, preservatives, etc. and/or other medicines.

The amount of Compound (I) or a pharmaceutically acceptable salt thereof that is contained in the pharmaceutical composition is not particularly limited and may be selected appropriately from a wide range, but normally Compound (I) or a salt thereof of the present invention is preferably contained in the amount of about 1% to about 70% of the pharmaceutical composition.

The method for administering the pharmaceutical composition of the present invention is not particularly limited, and it can be administered by a method suited to the dosage form, the age and sex of the subject/patient (especially, human), the disease status and other conditions. For example, it can be administered orally if it is in the form of a tablet, pill, liquid, suspension, emulsion, granules or capsules. If it is an injection, it can be administered intravenously either alone or in a mixture with an ordinary replacement fluid such as glucose or amino acids, or else it can be administered by itself intramuscularly, intradermally, subcutaneously or intraperitoneally as necessary. In the case of a suppository, it can be administered in the rectum.

The dose of the pharmaceutical composition may be selected appropriately according to the administration method, the age and sex of the subject/patient (especially, human), the disease status and other conditions, but normally about 0.001 to about 100 mg, preferably about 0.001 to about 50 mg per 1 kg of body weight can be administered per day in one to several portions.

The dose is affected by various conditions, and in some cases a dose below the aforementioned range may be sufficient, while in others a dose above the aforementioned range may be necessary.

The dose of the pharmaceutical composition includes, for example, a single dose of 0.15 mg, 0.3 mg, 0.6 mg, 1 mg, 2 mg, 4 mg, or 6 mg, but is not limited thereto.

The administration frequency of the above pharmaceutical composition includes, for example, twice a day, once a day, once in two days, once in three days, once in four days, once in five days, once in six days, once a week, or once in two weeks, or may be also a combination thereof. As an example of such combination administration, the pharmaceutical composition may be administered once on the first day, then after a week, once a day for five consecutive days.

Compound (I) of the present invention or a pharmaceutically acceptable salt thereof selectively inhibits the differentiation from hematopoietic stem cell toward megakaryocytes. Compound (I) of the present invention or a pharmaceutically acceptable salt thereof is useful for treating and/or preventing thrombocytosis, for example, essential thrombocythemia.

Disclosures of all patent literatures and non-patent literatures cited in the present description are incorporated by reference in the present description in their entireties.

### EXAMPLES

The present invention is explained in further detail below through the following Examples, but these do not limit the present invention, and these may be changed to the extent that they do not deviate from the scope of the present invention.

As the test substance,
(R)-6-(2,3-difluoro-4-(2-hydroxy-2-methylpropoxy)phenyl)-5-methyl-4,5-dihydropyridazin-3(2H)-one (Compound 1) was used.

### Example 1. Complex Formation of PDE3A and SLFN12

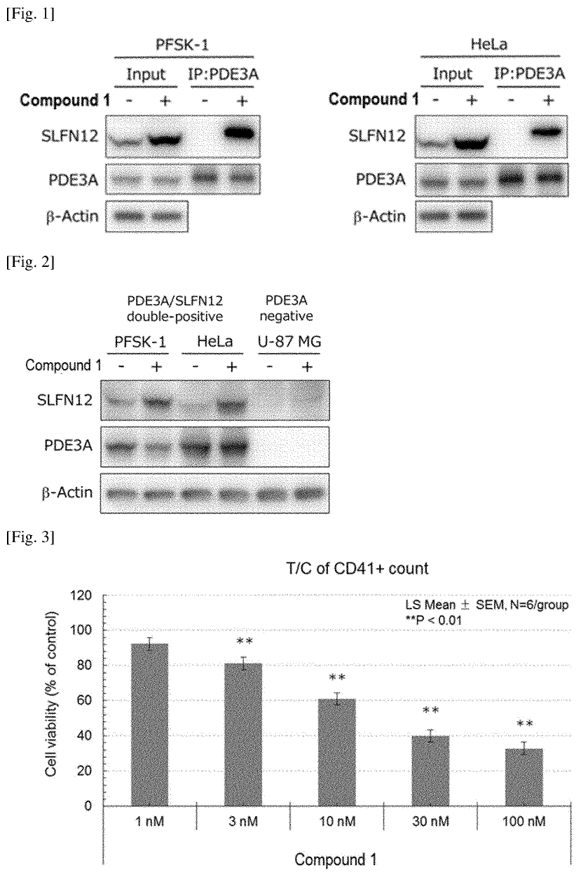

In order to examine whether Compound 1 forms a complex between PDE3A and SLFN12, a binding confirmation experiment using immunoprecipitation was performed. PFSK-1 and HeLa which are cell lines expressing PDE3A and SLFN12 were treated with Compound 1 (1 μmol/L) for 16 hours, and then immunoprecipitated using anti-PDE3A antibody. As a result, coprecipitation with SLFN12 was confirmed, as shown in FIG. 1.

### Example 2. Increase of SLFN12 Protein

In order to examine the effect of Compound 1 on the protein level of SLFN12, Western blot analysis was performed. PFSK-1, HeLa, or U-87 MG cell lines were treated with Compound 1 (1 μmol/L) for 16 hours, and SLFN12 protein in each test sample was detected.

The result is shown in FIG. 2. In the test samples using PFSK-1 or HeLa that highly expresses PDE3A, increased SLFN12 protein levels were observed when treated with Compound 1 compared to untreated with Compound 1. On the other hand, in the test samples using U-87 MG that hardly expresses PDE3A, no significant, Compound 1-dependent increase in SLFN12 protein level was observed.

### Example 3. Action of Inhibiting Megakaryocyte Differentiation

It was studied whether Compound 1 inhibits the differentiation from CD34-positive cells toward megakaryocytes. Bone marrow-derived CD34-positive cells were cultured for 10 days in the presence and absence of Compound 1 in megakaryocyte differentiation medium (HemaTox™ Megakaryocyte Kit, STEMCELL™ Technologies) according to the kit's instruction. The number of the CD41-positive megakaryocytes was counted by flow cytometry and the cell viability relative to the untreated cell group was calculated. The test was conducted three times using CD34-positive cells derived from different donors, and the cell viability was determined with the control as 100%.

The result is shown in FIG. 3. Compound 1 at concentrations of 3 nmol/L or higher significantly decreased the number of CD41-positive megakaryocytes. And, the 50% inhibitory concentration (IC₅₀) was 17.0 nmol/L (95% confidence interval: 10.3-23.7 nmol/L).

### Example 4. Action of Compound 1 to Differentiation Toward Erythroblastoid Cells

It was studied whether Compound 1 inhibits the differentiation from CD34-positive cells toward erythroblasts. Bone marrow-derived CD34-positive cells were cultured for 7 days in erythroid differentiation medium (HemaTox™ Erythroid Kit, STEMCELL™ Technologies) according to the kit's instruction. The number of the CD71-positive and CD235-positive erythroid cells were counted by flow cytometry and the cell viability relative to the untreated cell group was calculated. The test was conducted twice using CD34-positive cells derived from different donors, and the cell viability was determined with the control as 100%.

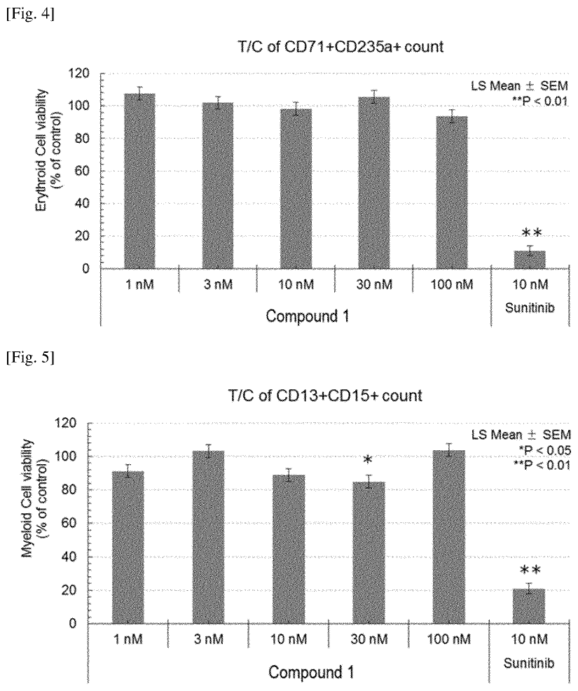

The result is shown in FIG. 4. Compound 1 at concentrations of 100 nmol/L or lower did not affect the differentiation from hematopoietic stem cells toward erythroid cells.

### Example 5. Action of Compound 1 to Differentiation Toward Myeloid Cells

It was studied whether Compound 1 inhibits the differentiation from CD34-positive cells toward myeloid cells. Bone marrow-derived CD34-positive cells were cultured for 7 days in myeloid differentiation medium (HemaTox™ Myeloid Kit, STEMCELL™ Technologies) according to the kit's instruction. The number of the CD13-positive and CD15-positive myeloid cells was counted by flow cytometry and the cell viability relative to the untreated cell group was calculated. The test was conducted twice using CD34-positive cells derived from different donors, and the cell viability was determined with the control as 100%.

The result is shown in FIG. 5. Compound 1 at 30 nmol/L inhibited the differentiation from hematopoietic stem cells toward myeloid cells, but there was no significant difference at 100 nmol/L, i.e., the result showed no concentration-dependent inhibitory effect of Compound 1 on the differentiation.

### INDUSTRIAL APPLICABILITY

The present compound or a pharmaceutically acceptable salt thereof inhibits the differentiation from hematopoietic stem cell toward megakaryocytes, thus it can be used for treating and/or preventing thrombocytosis such as essential thrombocythemia.

1: A pharmaceutical composition comprising a compound of formula (I): 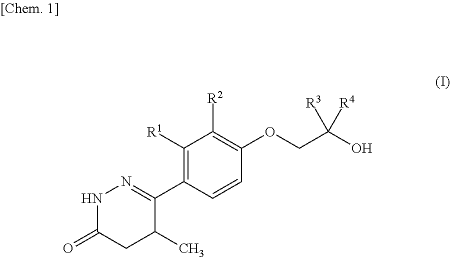
wherein R¹, R², R³, and R⁴ are independently halogen or C₁₋₃ alkyl,
or a pharmaceutically acceptable salt thereof, for preventing and/or treating a symptom and/or a disease related to enhanced induction of megakaryocyte differentiation.

2: The pharmaceutical composition of claim 1, wherein the compound of formula (I) is a compound of formula (Ia):

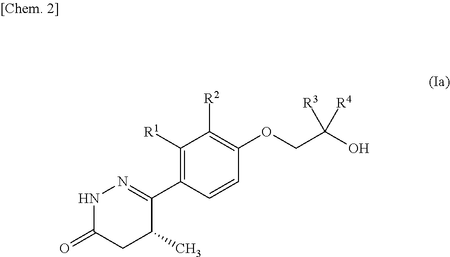

3: The pharmaceutical composition of claim 1, wherein R¹ and R² are fluorine, and R³ and R⁴ are methyl.

4: The pharmaceutical composition of claim 1, wherein the compound of formula (I) is 6-(2,3-difluoro-4-(2-hydroxy-2-methylpropoxy)phenyl)-5-methyl-4,5-dihydropyridazin-3(2H)-one or (R)-6-(2,3-difluoro-4-(2-hydroxy-2-methylpropoxy)phenyl)-5-methyl-4,5-dihydropyridazin-3(2H)-one.

5: The pharmaceutical composition of claim 1, wherein the symptom related to enhanced induction of megakaryocyte differentiation is increased platelet production.

6: The pharmaceutical composition of claim 1, wherein the disease related to enhanced induction of megakaryocyte differentiation is essential thrombocythemia.

7: Use of a compound of formula (I): 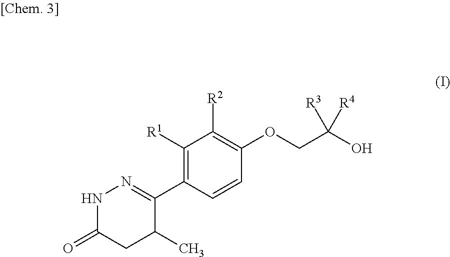
wherein R¹, R², R³, and R⁴ are independently halogen or C₁₋₃ alkyl,
or a pharmaceutically acceptable salt thereof, for preventing and/or treating a symptom and/or a disease related to enhanced induction of megakaryocyte differentiation.

8: A method for preventing and/or treating a symptom and/or a disease related to enhanced induction of megakaryocyte differentiation, comprising administering an effective amount of a compound of formula (I): 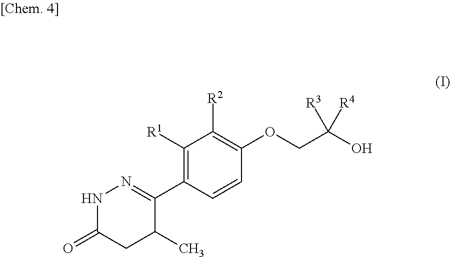
wherein R¹, R², R³, and R⁴ are independently halogen or C₁₋₃ alkyl,
or a pharmaceutically acceptable salt thereof to a subject in need thereof.

9: A compound of formula (I): 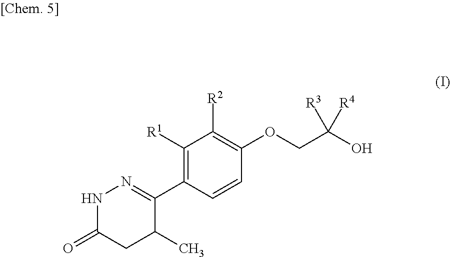
wherein R¹, R², R³, and R⁴ are independently halogen or C₁₋₃ alkyl,
or a pharmaceutically acceptable salt thereof for use in preventing and/or treating a symptom and/or a disease related to enhanced induction of megakaryocyte differentiation.

10: A method for manufacturing a medicament for preventing and/or treating a symptom and/or a disease related to enhanced induction of megakaryocyte differentiation, comprising the steps of:
providing a compound of formula (I): 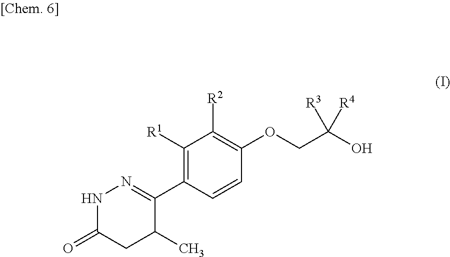
wherein R¹, R², R³, and R⁴ are independently halogen or C₁₋₃ alkyl,
or a pharmaceutically acceptable salt thereof, and
preparing a medicament with said compound or pharmaceutically acceptable salt thereof.
